(19) United States
(12) Patent Application Publication
(10) Pub. No.: US 2010/0004544 A1
(43) Pub. Date: Jan. 7, 2010

(54) OPTICAL TOMOGRAPHIC IMAGING PROBE, AND OPTICAL TOMOGRAPHIC IMAGING APPARATUS USING THE SAME

(76) Inventor: Masahiro TOIDA, Ashigarakami-gun (JP)

Correspondence Address: BIRCH STEWART KOLASCH & BIRCH, PO BOX 747, FALLS CHURCH, VA, 22040-0747, US

(21) Appl. No.: 12/496,375

(22) Filed: Jul. 1, 2009

(30) Foreign Application Priority Data
Jul. 2, 2008 (JP) 2008-173349

(51) Int. Cl. A61B 6/00

(52) U.S. Cl. 600/476

(57) ABSTRACT

To enable OCT imaging inside a blood vessel without having to block blood flow, through the use of an optical tomographic imaging probe including: a tubular probe outer casing; an optical fiber disposed inside the probe outer casing in an axial direction of the probe outer casing; a plurality of transparent inflatable/deflatable split balloons provided circumferentially across an outer circumferential surface of a transparent portion of the probe outer casing, through which a light beam is to be emitted from the optical fiber towards an measurement object, so as to equally divide the circumferential direction; and a balloon inflating/deflating device that respectively and individually inflates/deflates each split balloon, images are captured by removing only blood from imaging areas without having to block blood flow, and a tomographic image of the entire circumference of the inner wall of the blood vessel is obtained by compositing the images.

### BACKGROUND OF THE INVENTION

1. Field of the Invention

The present invention is related to an optical tomographic imaging probe and an optical tomographic imaging apparatus using the same, and in particular, to a technique for performing OCT imaging inside a blood vessel without having to block blood flow.

2. Description of the Related Art

Conventionally, there are known optical tomographic imaging apparatuses utilizing optical coherence tomography (OCT) measurement as a method of acquiring a tomographic image without dissecting a measurement object such as living tissue.

OCT measurement is an optical interferometric measurement method in which a light beam emitted from a light source is divided into two light beams, namely, a measurement light beam and a reference light beam, and which utilizes the fact that optical interference is only detected when respective optical path lengths of the measurement light beam and the reference light beam become consistent with each other within the range of a coherence length of the light source.

While OCT imaging technology has conventionally been used mainly in ophthalmology, recent studies have energetically been made on the use of OCT imaging technology in the photographing of vascular walls. However, in the case of blood vessels, due to the fact that a light beam used in OCT is significantly scattered by erythrocytes in a blood flow, there is a problem in that deterioration occurs in an image obtained by OCT imaging in an area where blood is present.

Therefore, in response thereto, methods have been proposed for, for example, performing OCT imaging by inflating a balloon inside a blood vessel to block blood flow and passing a normal saline solution to remove blood from a site to be photographed inside the blood vessel, or performing OCT imaging in a short period of time without specifically blocking blood flow by passing a normal saline solution at a burst so as to instantaneously secure an optical path with the flushing of the normal saline solution and rotating a probe at high speed (for example, refer to Benjamin, J. Vakoc, et. al., “Comprehensive esophageal microscopy by using optical frequency-domain imaging”, Gastrointestinal Endoscopy Vol. 65, No. 6898-905 (2007), Yaqoob, Zahid, et. al., “Methods and application area of endoscopic optical coherence tomography”, Journal of Biomedical Optics Vol. 11, No. 6, 063001 (2006), and the like).

FIGS. 9A to 9D show an OCT imaging method involving blocking blood flow and passing a normal saline solution.

FIG. 9A is a cross-sectional diagram of a blood vessel showing a state in which a probe is inserted into the blood vessel; FIG. 9B is a cross-sectional diagram of the probe showing the vicinity of a balloon provided on the probe so as to block blood flow; FIG. 9C is a cross-sectional diagram of the probe taken perpendicular to a length direction thereof, and FIG. 9D is a cross-sectional diagram of a blood vessel showing a situation in which the balloon is inflated to block blood flow and a normal saline solution is passed.

As shown in FIG. 9A, a probe 110 is inserted into a blood vessel 100. A balloon 112 for blocking blood flow is provided on the probe 110. In addition, although not shown, an imaging section that irradiates a light beam on a vascular wall and receives a reflected light beam thereof is disposed on the probe 110 to the left of the balloon 112. Note that in FIG. 9(a), the balloon 112 has not been inflated and blood 102 flows from right to left in the drawing.

As shown in FIGS. 9B and 9C, the probe 110 is provided with the balloon 112. The balloon 112 is arranged so as to be inflated and to block blood flow due to air 4supplied from an air supplying path 114 via an air supplying port 114a. In addition, an ejecting port 116a for ejecting a normal saline solution supplied from a normal saline solution supplying path 116 into a blood vessel is provided on the probe 110 on a tip side (downstream-side of the blood flow) of the balloon 112. An optical fiber 120 is disposed at the center of the probe 110.

As shown in FIG. 9D, during OCT imaging, air is supplied to the balloon 112 to inflate the same, and a surface of the balloon 112 is brought into close contact with an inner wall of the blood vessel 100 to block the flow of blood 102. Subsequently, a normal saline solution 117 is ejected from the ejecting port 116a into the blood vessel to remove blood of a site to be photographed, whereby OCT imaging is performed.

Furthermore, FIGS. 10A to 10C show a method in which a normal saline solution is flushed without blocking blood flow to instantaneously secure a field of view and a probe is rotated at high speed, whereby OCT imaging is performed while the field of view is being secured.

FIG. 10A is a diagram showing a probe to be used in the method described above; FIG. 10B is a cross-sectional diagram of the probe taken perpendicular to a length direction thereof; and FIG. 10C is a cross-sectional diagram of a blood vessel showing a situation in which OCT imaging is performed by passing a normal saline solution at a burst.

As shown in FIGS. 10A and 10B, a probe 130 in this case includes: a normal saline solution supplying path 132 for supplying a normal saline solution to the outside; and an ejecting port 132a for ejecting a normal saline solution into a blood vessel. An optical fiber 140 is provided at the center of the probe 130.

As shown in FIG. 10C, when performing OCT imaging, the normal saline solution 117 is ejected into the blood vessel at a burst from the ejecting port 132a to instantaneously secure a field of view by the flushing of the normal saline solution 117, whereby imaging is promptly performed while the field of view is being secured. Although a normal probe makes about 10 rotations per second, in this case, the probe is rotated at high speed to make about 50 rotations per second, whereby imaging is performed at high speed. In the drawing, it is assumed that blood flows from the right to the left.

However, with the conventional method of performing OCT imaging by blocking blood flow and securing an optical path by flushing a normal saline solution, problematically, there is a concern that examination risks may increase because the blocking of blood flow places a greater burden on a patient. On the other hand, while the method of securing an optical path only for a short period of time by flushing a normal saline solution without blocking blood flow and performing OCT imaging at high speed within that period of time requires increased speeds at which imaging of several hundred frames is performed per second, realizing such a high-speed rotation with an intravascular probe insertable into a blood vessel is difficult.

### SUMMARY OF THE INVENTION

The present invention has been made in consideration of such circumstances, and an object thereof is to provide an optical tomographic imaging probe that enables OCT imaging to be performed inside a blood vessel without having to block blood flow, and an optical tomographic imaging apparatus using the same.

In order to achieve the object described above, a first aspect of the present invention provides an optical tomographic imaging probe including: a tubular probe outer casing; an optical fiber disposed inside the probe outer casing in an axial direction of the probe outer casing; a plurality of transparent inflatable/deflatable split balloons provided circumferentially across an outer circumferential surface of a transparent portion of the probe outer casing, through which a light beam is to be emitted from the optical fiber towards an measurement object, so as to equally divide outer periphery area; and a balloon inflating/deflating device that respectively and individually inflates/deflates each split balloon.

As shown, since a balloon provided on an outer circumferential portion of a probe is split into a plurality of balloons to be respectively and independently inflated/deflated, OCT photography can now be performed without having to block blood flow even when imaging is carried out inside a blood vessel.

In addition, in order to achieve the object described above, a second aspect of the present invention provides an optical tomographic imaging apparatus including: a light source that emits a light beam; a light splitting device that splits the light beam emitted from the light source into a measurement light beam and a reference light beam; an optical path length adjusting device that adjusts an optical path length of the reference light beam split by the light splitting device; an irradiating optical system that irradiates the measurement light beam split by the light splitting device onto a measurement object; an optical multiplexing device that multiplexes a reflected light beam from the measurement object when the measurement light beam is irradiated on the measurement object with the reference light beam; an interference light detecting device that detects an interference light beam of the multiplexed reflected light beam and reference light beam; and an image acquiring device that acquires a tomographic image of the measurement object from the detected interference light beam, wherein the irradiating optical system includes the optical tomographic imaging probe according to the first aspect, and the optical tomographic imaging apparatus is arranged such that the optical tomographic imaging probe is inserted into a blood vessel as the measurement object, a portion of the plurality of split balloons are inflated to cause the outer surfaces of the inflated split balloons to come into close contact with an inner wall of the blood vessel to remove blood from the close-contact portions while split balloons other than the inflated split balloons are deflated so as to provide a gap between the outer surfaces of the deflated split balloons and the inner wall of the blood vessel to secure blood flow, the measurement light beam is irradiated onto the inner wall of the blood vessel via the split balloons in close contact with the inner wall of the blood vessel to acquire images of portions of the inner wall of the blood vessel in close contact with the outer surfaces of the inflated split balloons, switching is subsequently performed between the inflated split balloons and the deflated split balloons, images of portions of the inner wall of the blood vessel in close contact with the outer surfaces of split balloons inflated after the switching are acquired, and images obtained before and after the switching are composited by the image acquiring device to obtain an image of the entire circumference of the inner wall of the blood vessel.

Accordingly, OCT photography can be performed without having to block blood flow and a tomographic image of an entire circumference of an inner wall of a blood vessel can now be obtained.

Furthermore, according to a third aspect of the present invention, the split balloons are split into an even number of split balloons of four or greater, the split balloons are separated into two groups respectively made up of every other split balloon in the circumferential direction, and the inflation/deflation of the split balloons is switched between the two groups by the balloon inflating/deflating device.

Accordingly, when inflating a split balloon, pressure can now be applied evenly in the circumferential direction of the blood vessel.

In addition, according to a fourth aspect of the present invention, the balloon inflating/deflating device includes: an air supplying pump that supplies air to each of the split balloons via an air supplying path provided in the optical tomographic imaging probe; and an air supply switching device that switches the supply of air to among the respective split balloons.

Furthermore, according to a fifth aspect of the present invention, the balloon inflating/deflating device includes: a liquid supplying pump that supplies a normal saline solution to each of the split balloons via a liquid supplying path provided in the optical tomographic imaging probe; and a liquid supply switching device that switches the supply of the normal saline solution to among the respective split balloons.

As shown, various devices can be applied to inflate balloons.

As described above, according to the present invention, since a balloon provided on an outer circumferential portion of a probe is split into a plurality of balloons to be respectively and independently inflated/deflated, OCT photography can now be performed without having to block blood flow even when performing imaging inside a blood vessel. In addition, by compositing images captured separately, a tomographic image of an entire circumference of an inner wall of a blood vessel can be obtained without having to block blood flow and therefore without placing a burden on the object to be examined.

### BRIEF DESCRIPTION OF THE DRAWINGS

FIG. 1 is a schematic configuration diagram showing an embodiment of an optical tomographic imaging apparatus according to the present invention;

FIG. 2A is a longitudinal cross-sectional diagram of a probe according to the present embodiment showing an enlargement of a tip of the probe, while FIG. 2B is a cross-sectional diagram of the probe taken perpendicular to the longitudinal direction thereof;

FIG. 3A is a longitudinal cross-sectional diagram of a blood vessel into which the probe is inserted, while FIG. 3B is a cross-sectional diagram of the blood vessel taken perpendicular to the longitudinal direction thereof;

FIG. 4A is, similarly, a longitudinal cross-sectional diagram of a blood vessel into which the probe is inserted, while FIG. 4B is a cross-sectional diagram of the blood vessel taken perpendicular to the longitudinal direction thereof;

FIG. 5 is a cross-sectional diagram showing a split state of a balloon;

FIG. 6 is an explanatory diagram showing how an air supply switching valve and an air supplying pump are controlled;

FIGS. 7A and 7B are explanatory diagrams showing inflation/deflation of a split balloons;

FIG. 8 is an explanatory diagram showing how an image of an entire circumference is composited from split images acquired through inflation/deflation control of the respective split balloons;

FIGS. 9A to 9D are diagrams showing how OCT imaging is conventionally performed by blocking blood flow and passing a normal saline solution, wherein FIG. 9A is a longitudinal cross-sectional diagram of a blood vessel showing a state in which a probe is inserted into the blood vessel, FIG. 9B is a longitudinal cross-sectional diagram of the probe showing the vicinity of a balloon provided thereon, FIG. 9C is a cross-sectional diagram of the probe taken perpendicular to the longitudinal direction thereof; and FIG. 9D is a longitudinal cross-sectional diagram of a blood vessel showing a state in which OCT imaging is performed by inflating the balloon to block blood flow; and

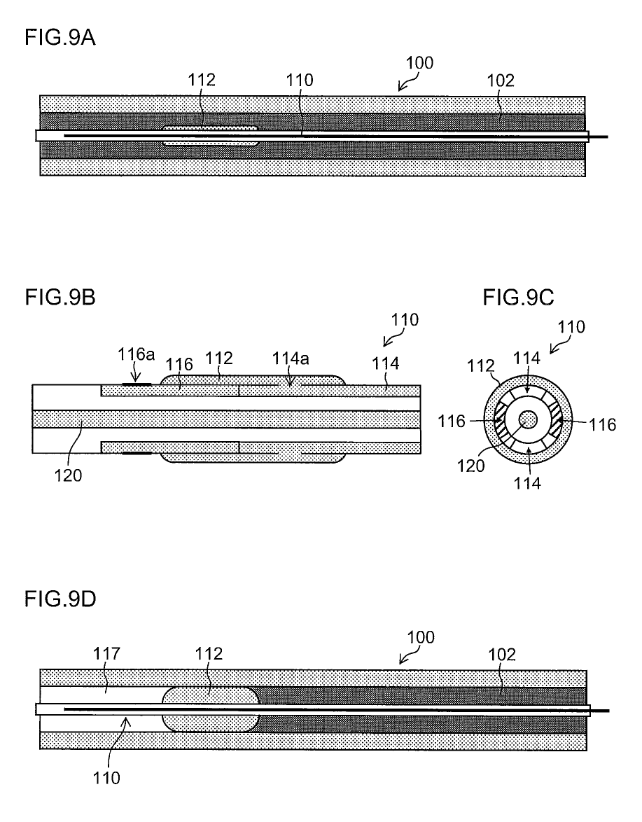

FIGS. 10A to 10C are diagrams showing how conventional OCT imaging is performed by securing a field of view with a flush of a normal saline solution without having to block blood flow, wherein FIG. 10A is a longitudinal cross-sectional diagram of a probe; FIG. 10B is a cross-sectional diagram of the probe taken perpendicular to the longitudinal direction thereof; and FIG. 10C is a longitudinal cross-sectional diagram of a blood vessel showing how OCT imaging is performed by flushing a normal saline solution.

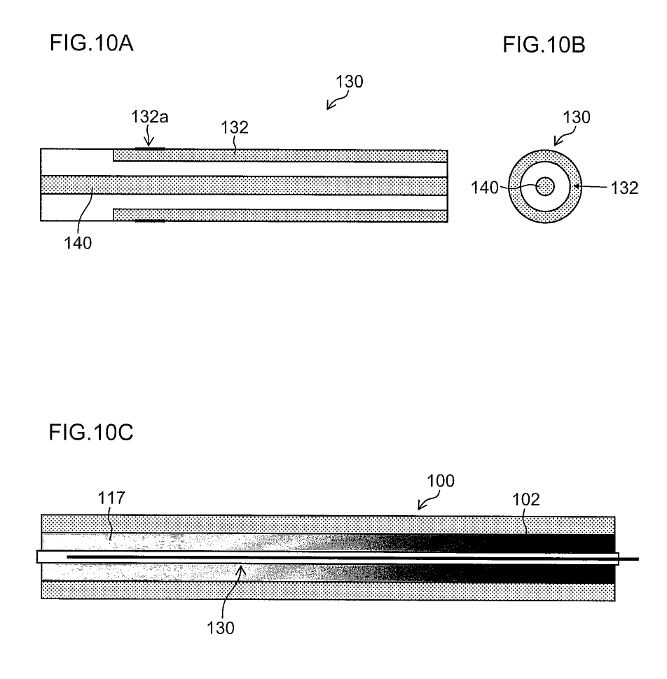

### DETAILED DESCRIPTION OF THE PREFERRED EMBODIMENTS

An optical tomographic imaging probe and an optical tomographic imaging apparatus using the same according to the present invention will now be described in detail with reference to the attached drawings.

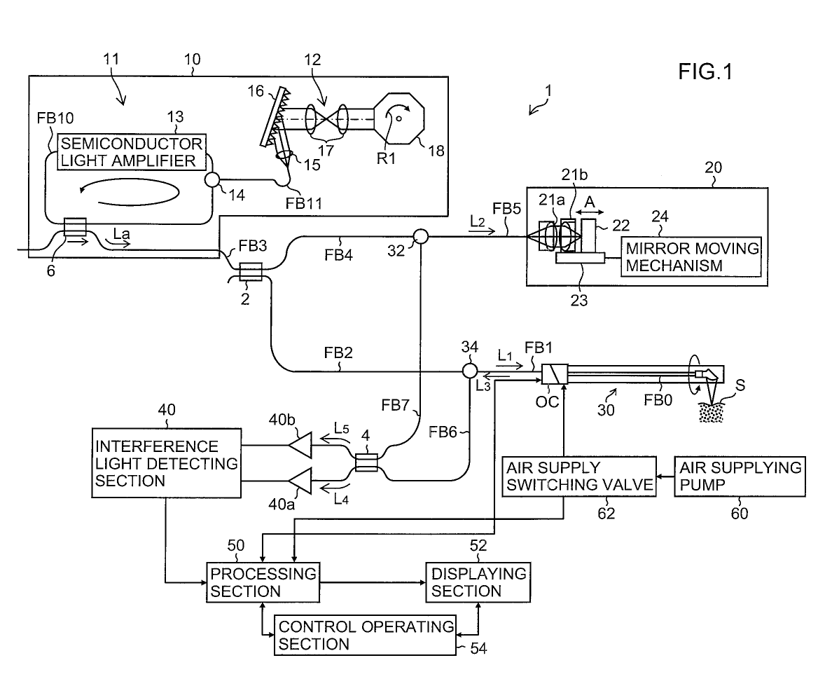

FIG. 1 is a schematic configuration diagram showing an overall configuration of an embodiment of an optical tomographic imaging apparatus according to the present invention.

As shown in FIG. 1, an optical tomographic imaging apparatus 1 according to the present embodiment is arranged so as to acquire a tomographic image of a blood vessel through SS-OCT (swept source OCT) measurement. The optical tomographic imaging apparatus 1 includes: a light source unit 10 that emits light beams; an optical path length adjusting device 20 that adjusts an optical path length of a reference light beam L2 split by a light splitting device 2 which splits a light beam La emitted from the light source unit 10 into a measurement light beam L1 and the reference light beam L2; a probe 30 that guides the measurement light beam L1 split by the light splitting device 2 to a measurement object S; an optical multiplexing device 4 that multiplexes a reflected light beam L3 reflected off of the measurement object S when the measurement light beam L1 from the probe 30 is irradiated thereon with the reference light beam L2; an interference light detecting section 40 that detects an interference light beam L4 obtained by multiplexing the reflected light beam L3 and the reference light beam L2 with the optical multiplexing device 4; a processing section (image acquiring device) 50 that detects an intensity of the interference light beam L4 at each depth position of the measurement object 3 and acquires a tomographic image of the measurement object S by analyzing the frequency of an interference signal detected by the interference light detecting section 40; a control operating section 54 that controls a displaying section 52 for displaying an acquired tomographic image as well as the respective sections, and the like.

Moreover, the present embodiment is arranged so as to capture a tomographic image of specifically a blood vessel as the measurement object S. Therefore, while a detailed description will be given later, in order to enable OCT imaging of the inside of a blood vessel without having to block blood flow, a balloon (to be described later) as a blood flow blocking device that partially blocks blood flow is provided around a probe outer casing on which is disposed an optical lens that emits the measurement light beam L1 to the measurement object S at a tip of the probe 30. Furthermore, also provided are: an air supplying pump 60 for supplying air to and inflating the balloon; and an air supply switching valve 62.

The light source unit 10 is arranged so as to emit a laser beam La while sweeping frequencies at regular periods. To this end, the light source unit 10 includes: a light source 11 that emits a light beam having a certain wavelength band; and a wavelength selecting device 12 that selects a wavelength emitted from the light source 11. The light source 11 is made up of a semiconductor light amplifier (semiconductor gain medium) 13, connected in a loop configuration to an optical fiber FB10, which emits a spontaneously emitted light beam and amplifies a spontaneously emitted light beam guided from the optical fiber FB10. The light source 11 functions to emit a spontaneously emitted light beam to the side of one end of the optical fiber FB10 in response to the injection of a drive current, and to amplify a light beam that enters from the side of the other end of the optical fiber FB10. In addition, when the drive current is supplied to the semiconductor light amplifier 13, the laser beam La is to be emitted to an optical fiber FB11 from a laser light source resonator formed by the semiconductor light amplifier 13 and the optical fiber FB10.

The wavelength selecting device 12 selects a wavelength of a spontaneously emitted light beam guided from the optical fiber FB10 as a wavelength sweeping light source filter, and is arranged so that a spontaneously emitted light beam enters via the optical fiber FB11 from an optical bifurcator (circulator) 14 coupled to the optical fiber FB10. The wavelength selecting device 12 includes: a collimator lens 15; a diffraction grating element 16; an optical system (optical face angle error correcting lens) 17; a rotary polygon mirror (polygon mirror) 18, and the like.

A light beam entering from the optical fiber FB11 is reflected by the rotary polygon mirror 18 via the collimator lens 15, the diffraction grating element 16, and the optical system 17. The reflected light beam reenters the optical fiber FB11 via the optical system 17, the diffraction grating element 16, and the collimator lens 15.

The rotary polygon mirror (polygon mirror) 18 is arranged so as to rotate in the direction of arrow R1 such that the angle of each reflecting face thereof changes with respect to an optical axis of the optical system 17. Accordingly, only light beams of a specific frequency range among light beams separated at the diffraction grating element 16 returns to the optical fiber FB11.

The frequency of the light beam that returns to the optical fiber FB11 is determined by an angle formed by the optical axis of the optical system 17 and a reflecting face. Light beams of a specific frequency range having entered the optical fiber FB11 enters the optical fiber FB10 from the optical bifurcator 14 and, as a result, the laser beam La of a specific frequency range is emitted to the side of an optical fiber FB3 from an optical fiber coupler 6.

Therefore, when the rotary polygon mirror 18 rotates at a constant speed in the direction of arrow R1, the wavelength of the light beam reentering the optical fiber FB11 is to be swept at regular periods. In other words, the laser beam La whose wavelength is swept at regular periods is emitted from the light source unit 10 to the side of the optical fiber FB3 via the optical fiber coupler 6.

The light splitting device 2 is made up of, for example, 2×2 optical fiber couplers, and is arranged so as to split the laser beam La guided from the light source unit 10 via the optical fiber FB3 into the measurement light beam L1 and the reference light beam L2. The light splitting device 2 is optically connected to two optical fibers FB2 and FB4 respectively, whereby the measurement light beam L1 is to be guided to the side of the optical fiber FB2 while the reference light beam L2 is to be guided to the side of the optical fiber FB4.

One tip of the optical fiber FB4 is connected to an optical bifurcator (circulator) 32. An optical fiber FB5 and an optical fiber FB7 are further connected to the optical bifurcator 32. The reference light beam L2 guided from the optical fiber FB4 is guided to the optical fiber FB5 from the optical bifurcator 32. Moreover, the optical path length adjusting device 20 is disposed ahead of the optical fiber FB5.

The optical path length adjusting device 20 is arranged so as to vary the optical path length of the reference light beam L2 in order to adjust the position where the acquisition of a tomographic image is to be commenced. The optical path length adjusting device 20 includes: a reflecting mirror 22 that reflects the reference light beam L2 emitted from the optical fiber FB5; a first optical lens 21a disposed between the reflecting mirror 22 and the optical fiber FB5; and a second optical lens 21b disposed between the first optical lens 21a and the reflecting mirror 22.

The first optical lens 21a functions to convert the reference light beam L2 emitted from the optical fiber FB5 into a parallel light beam, and to collect the reference light beam L2 reflected off of the reflecting mirror 22 into the core of the optical fiber FB5. In addition, the second optical lens 21b functions to collect the reference light beam L2 converted into a parallel light beam by the first optical lens 21a onto the reflecting mirror 22 and to convert the reference light beam L2 reflected off of the reflecting mirror 22 into a parallel light beam.

Accordingly, the reference light beam L2 emitted from the optical fiber FB5 is converted into a parallel light beam by the first optical lens 21a and collected onto the reflecting mirror 22 by the second optical lens 21b. Subsequently, the reference light beam L2 reflected off of the reflecting mirror 22 is converted into a parallel light beam by the second optical lens 21b and collected into the core of the optical fiber FB5 by the first optical lens 21a.

Furthermore, the optical path length adjusting device 20 includes: a movable stage 23 that fixes the second optical lens 21b and the reflecting mirror 22; and a mirror moving mechanism 24 that moves the movable stage 23 in the direction of the optical axis of the first optical lens 21a. The optical path length of the reference light beam L2 varies in correspondence with the movement of the movable stage 23 in the direction of arrow A.

A light beam whose optical path length has been changed by the optical path length adjusting device 20 reenters the optical fiber FB5, and is further guided to the side of the optical fiber FB7 via the optical bifurcator 32.

On the other hand, an optical bifurcator (circulator) 34 is connected ahead of the optical fiber FB2 that guides the measurement light beam L1. An optical fiber FB1 and an optical fiber FB6 are further connected to the optical bifurcator 34, whereby the measurement light beam L1 is guided to the side of the optical fiber FB1 from the optical bifurcator 34.

The probe 30 is optically connected to one tip of the optical fiber FB1, whereby the measurement light beam L1 is to be guided from the optical fiber FB1 to an optical fiber FB0 inside the probe 30. The probe 30 is arranged so as to be, for example, inserted into a blood vessel via a forceps channel from a forceps opening, and is removably mounted to the optical fiber FB1 by an optical connector OC.

The probe 30 is connected to the optical fiber FB1 via the optical connector OC, whereby the measurement light beam L1 guided by the optical fiber FB1 enters the optical fiber FB0 inside the probe 30. The measurement light beam L1 having entered the optical fiber FB0 is transmitted by the same and is irradiated on the measurement object S (vascular wall). In addition, the returning light beam (reflected light beam) L3 reflected off of the measurement object S is arranged so as to enter the optical fiber FB0, thereby enabling OCT imaging of the vascular wall to be performed.

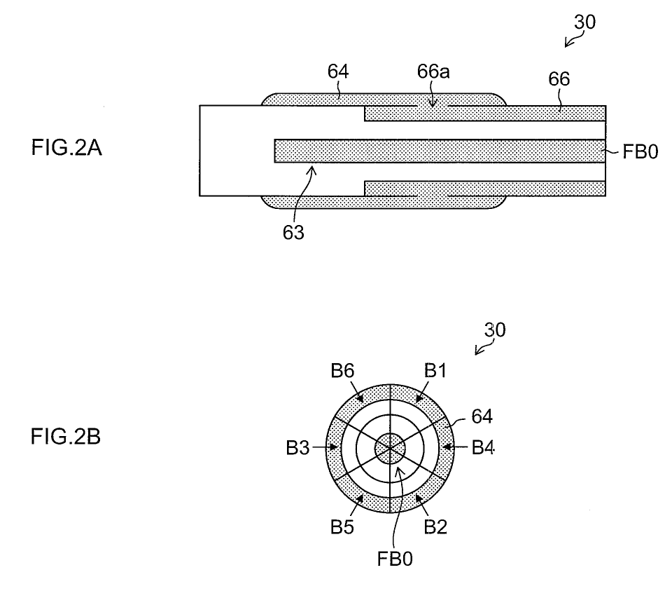

The probe 30 that is an optical tomographic imaging probe which is a feature of the present invention and which is ingeniously designed so as to enable OCT imaging to be performed inside a blood vessel without having to block blood flow will now be described in detail with reference to FIGS. 2A to 4B.

FIG. 2A is a longitudinal cross-sectional diagram of the probe 30 showing an enlargement of a tip thereof, while FIG. 2B is a cross-sectional diagram of the probe 30 taken perpendicular to the longitudinal direction thereof.

As shown in FIG. 2A, a balloon 64 that is inflated and which blocks blood flow upon introduction of air is provided on the outer circumference of the tip of the probe 30. In addition, the probe 30 is provided with an air supplying path 66 for supplying air to the balloon 64 and an air supplying port 66a through which air is introduced to the balloon 64 from the air supplying path 66. Furthermore, the optical fiber FB0 that guides the measurement light beam L1 is disposed at the center of the probe 30 and, although not shown, an optical lens or the like which irradiates the measurement light beam L1 guided from the optical fiber FB0 to a vascular wall that is the measurement object S, collects the reflected light beam L3 thereof, and causes the reflected light beam L3 to enter the optical fiber FB0, is provided on a tip 63 of the probe 30.

The measurement light beam L1 is irradiated on a vascular wall from the optical fiber FB0 via the balloon 64, and the reflected light beam L3 thereof also enters the optical fiber FB0 via the balloon 64. To this end, the balloon 64 is formed of, for example, a transparent substance so as to allow transmission of light. In addition, to ensure that blood does not exist between the measurement light beam L1 and a vascular wall during imaging, the balloon 64 must be arranged so as to be inflated in such a manner that the surface of the balloon 64 comes completely into close contact with an imaging area of the vascular wall and that blood is removed therefrom. Furthermore, at this point, instead of completely blocking blood flow, blood flow is to be secured for portions other than the imaging area so as to avoid burdening the patient (the object to be examined).

To this end, the balloon 64 is split into a plurality of portions in a circumferential direction thereof, whereby each split portion is arranged so as to be independently inflatable/deflatable. In the example shown in FIG. 2B, the balloon 64 is circumferentially split into six equal portions. The respective split balloons B1, B2, B3, B4, B5 and B6, resulting from splitting the balloon 64 into six equal portions, are each independently inflatable/deflatable.

However, at this point, a deviation of a split balloon to be inflated towards one side may cause a movement or disfigurement of the blood vessel, thereby preventing an accurate image from being captured.

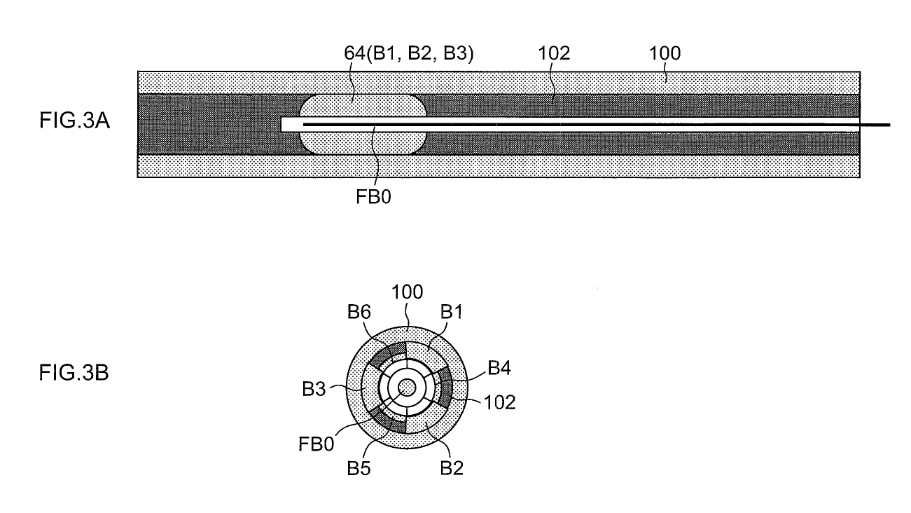

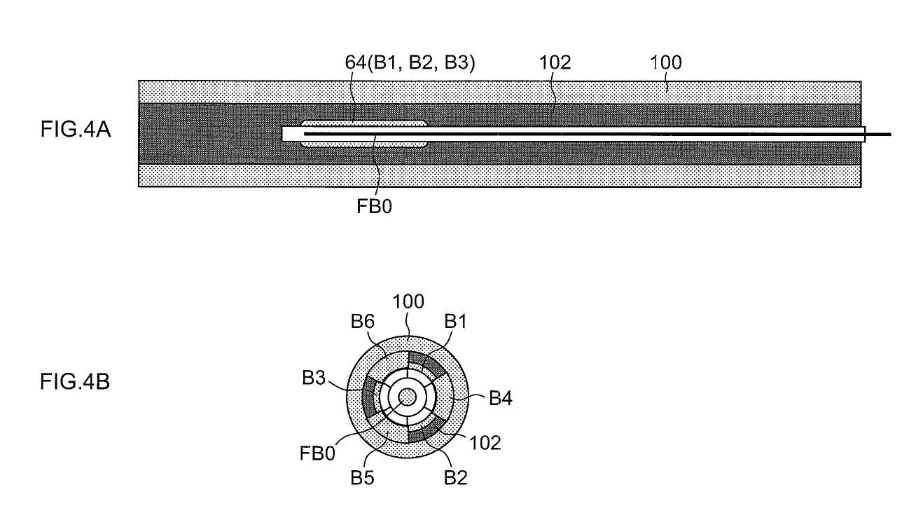

In consideration thereof, as shown in FIGS. 3A to 4B, it is preferable that every other split balloon of the six split balloons B1, B2, B3, B4, B5 and B6 is alternately inflated so that pressure is applied as evenly as possible in the circumferential direction of the blood vessel.

FIG. 3A is a longitudinal cross-sectional diagram of the blood vessel 100 into which the probe 30 is inserted, while FIG. 3B is a cross-sectional diagram of the blood vessel 100 taken perpendicular to the longitudinal direction thereof.

As shown in FIG. 3B, first, the split balloons B1, B2, and B3 are inflated while the split balloons B4, B5, and B6 are deflated. At this point, as shown in FIG. 3A, the split balloons B1, B2, and B3 are inflated so as to include the tip of the optical fiber FB0, whereby the surfaces thereof come into close contact with the wall of the blood vessel 100 and remove blood from the portions in close contact.

Since the balloon 64 (split balloons B1, B2, B3, B4, B5 and B6) is formed by a transparent member so as to enable transmission of light, in areas where the surfaces of the split balloons B1, B2, and B3 are in close contact with the wall surface of the blood vessel 100 and blood is removed therefrom, a light beam emitted from the optical fiber FB0 is able to irradiate a wall face of the blood vessel 100 without being scattered by erythrocytes in the blood.

Accordingly, by rotating the optical fiber FB0, images of every other area of the wall face of the blood vessel 100 in close contact with the split balloons B1, B2, and B3 are captured.

In addition, at this point, as shown in FIG. 3B, since the split balloons B4, B5, and B6 are in a deflated state, gaps are formed between the surfaces of the split balloons B4, B5, and B6 and the wall face of the blood vessel 100, thereby securing blood flow and reducing the impact on the object to be examined.

Furthermore, FIGS. 4A and 4B are similar to FIGS. 3A and 3B, wherein FIG. 4A is a longitudinal cross-sectional diagram of the blood vessel 100 into which the probe 30 is inserted, and FIG. 4B is a cross-sectional diagram of the blood vessel 100 taken perpendicular to the longitudinal direction thereof.

Next, as shown in FIG. 4B, the split balloons B4, B5, and B6 are inflated while the split balloons B1, B2, and B3 are deflated. At this point, as shown in FIG. 4A, the split balloons B4, B5, and B6 are inflated so as to include the tip of the optical fiber FB0, whereby the surfaces thereof come into close contact with the wall of the blood vessel 100 and remove blood from the portions in close contact.

In areas where the surfaces of the split balloons B4, B5, and B6 come into close contact with the wall of the blood vessel 100 and remove blood therefrom, a light beam emitted from the optical fiber FB0 is able to irradiate the wall face of the blood vessel 100 without being scattered by erythrocytes in the blood. Accordingly, by rotating the optical fiber FB0, images of every other area of the wall face of the blood vessel 100 in close contact with the split balloons B4, B5, and B6 are captured.

In addition, at this point, as shown in FIG. 4B, since the split balloons B1, B2, and B3 are in a deflated state, gaps are formed between the surfaces of the split balloons B1, B2, and B3 and the wall face of the blood vessel 100, thereby securing blood flow and reducing the impact on the object to be examined.

In this manner, images are obtained for every other area among six circumferentially equally split areas of the wall face of the blood vessel 100 with which the balloon 64 is in close contact. By compositing these images, an image of the entire circumferential direction of the wall face of the blood vessel 100 can be obtained.

While the examples shown in FIGS. 2A to 4B have been arranged so that the balloon 64 is inflated by supplying air thereto, the material used for inflating the balloon 64 need not be limited to air. For example, the balloon 64 may alternatively be arranged so as to be inflated using a normal saline solution by replacing the air supplying path 66 with a liquid supplying path and supplying the normal saline solution instead of air.

Returning once again to FIG. 1, the reflected light beam L3 having entered the optical fiber FB0 is arranged so as to be emitted from the optical fiber FB0 to the optical fiber FB1 via the optical connector OC.

The reflected light beam L3 having entered the optical fiber FB1 is to be guided to the side of the optical fiber FB6 via the optical bifurcator 34. Meanwhile, the reference light beam L2 whose optical path length has been changed by the optical path length adjusting device 20 is guided to the side of the optical fiber FB7 via the optical fiber FB5 and the optical bifurcator 32.

The reflected light beam L3 guided by the optical fiber FB6 and the reference light beam L2 guided by the optical fiber FB7 are multiplexed by the optical multiplexing device 4 and are outputted as interference light beams L4 and L5. The interference light beam L4 is arranged so as to enter a detector 40a, while the interference light beam L5 is arranged so as to enter a detector 40b.

The interference light detecting section 40 is arranged so as to detect the interference light beams L4 and L5 generated by multiplexing the reflected light beam L3 and the reference light beam L2 as interference signals. In addition, the interference light detecting section 40 functions to adjust the balance between respective intensities of the interference light beams L4 and L5 outputted from the optical multiplexing device 4 based on the detection results of the detectors 40a and 40b.

The processing section 50 detects an area at a measurement position in which the probe 30 and the measurement object S are in contact with each other or, more precisely, an area at which a surface of the probe outer casing of the probe 30 and a surface of the measurement object S are in contact with each other from an interference signal detected by the interference light detecting section 40. In addition, the processing section 50 acquires a tomographic image from an interference signal detected by the interference light detecting section 40.

The displaying section 52 is made up of a CRT, a liquid crystal display device, or the like, and displays a tomographic image transmitted from the processing section 50.

The control operating section 54 includes: an input device such as a keyboard or a mouse; and a control device that manages various conditions based on inputted information, and is connected to the processing section 50 and the displaying section 52. Based on an operator's instruction inputted via the input device, the control operating section 54 performs inputting, setting, and changing of various processing conditions and the like at the processing section 50 as well as changing and the like of display settings of the displaying section 52.

Operations of the optical tomographic imaging apparatus 1 according to the present embodiment will be described below.

First, by moving the base (movable stage) 23 in the direction of arrow A using the mirror moving mechanism 24 of the optical path length adjusting device 20 shown in FIG. 1, an optical path length is adjusted and set so that the measurement object S is positioned in the measurable region.

Subsequently, the laser beam La is emitted from the light source unit 10. The emitted laser beam La is split into the measurement light beam L1 and the reference light beam L2 by the light splitting device 2. The measurement light beam L1 is guided from the optical fiber FB2 to the optical connector OC via the optical bifurcator 34 and the optical fiber FB1. In addition, the measurement light beam L1 is guided from the optical connector OC to the optical fiber FB0 inside the probe 30.

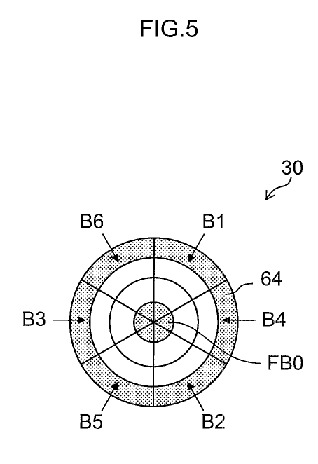

As shown in FIG. 5, the balloon 64 provided on the tip of the probe 30 is circumferentially divided into six equal parts, namely, the split balloons B1, B2, B3, B4, B5, and B6. In the state shown in FIG. 5, all of the split balloons B1, B2, B3, B4, B5, and B6 are in a deflated state. The inflation/deflation of the respective split balloons B1, B2, B3, B4, B5, and B6 is to be controlled using the air supplying pump 60 and the air supply switching valve 62 as described below.

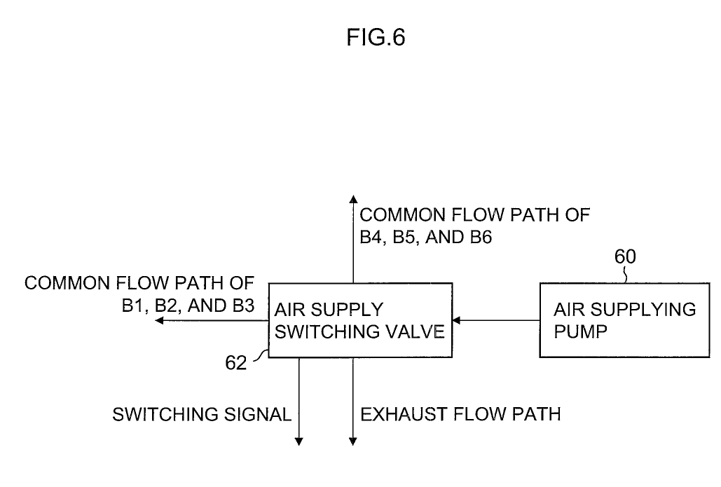

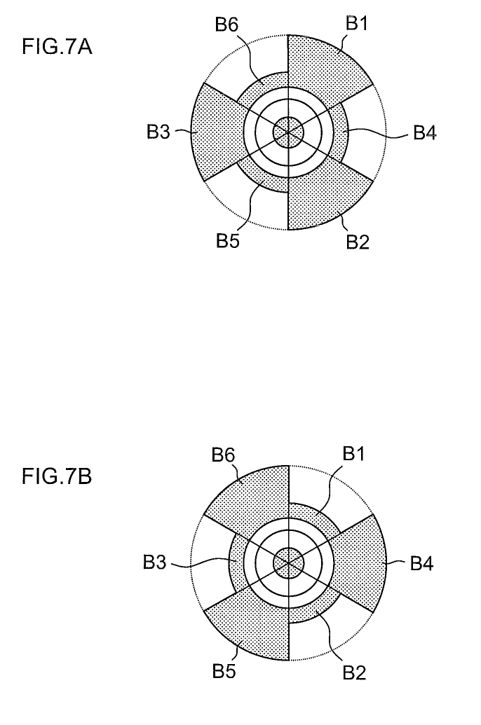

As shown in FIG. 6, when switching the air supply switching valve 62 to the side of a common flow path of the split balloons B1, B2, and B3 and supplying air with the air supplying pump 60, air is delivered from the air supplying pump 60 to the split balloons B1, B2, and B3, thereby inflating the split balloons B1, B2, and B3 as shown in FIG. 7A. At this point, as shown in FIG. 7A, the split balloons B4, B5, and B6 are still in a deflated state.

The surfaces of the inflated split balloons B1, B2, and B3 are brought into close contact with the wall face of the blood vessel 100, and blood is removed from the close-contact portions. The balloon 64 (split balloons B1, B2, and B3) are formed of a transparent material, and since the inside of the balloon 64 is only filled with air, the balloon 64 is capable of transmitting light. Accordingly, by emitting the measurement light beam L1 while rotating the optical fiber FB0, the measurement light beam L1 is transmitted through the balloon 64 (split balloons B1, B2, and B3) and irradiates the wall face of the blood vessel 100 with which the split balloons B1, B2, and B3 are in close contact. At this point, since blood has been removed from these portions, the measurement light beam L1 irradiating the wall face of the blood vessel 100 is not scattered by blood.

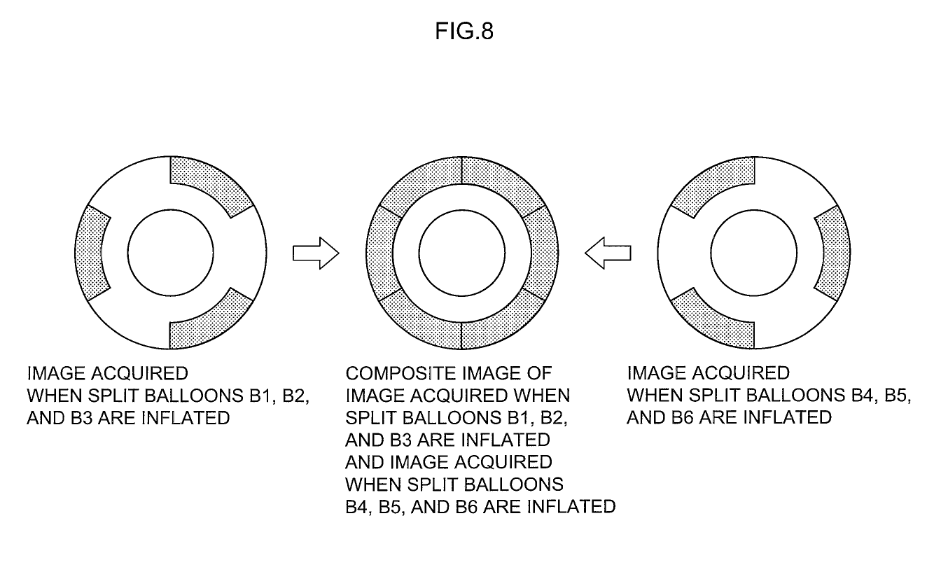

In addition, the optical fiber FB0 is in rotation inside the probe 30. By rotating the optical fiber FB0 while emitting the measurement light beam L1 therefrom, as depicted in the left side of FIG. 8, images are acquired for every other area corresponding to portions with which the inflated (expanded) split balloons B1, B2, and B3 are in close contact among six circumferentially equally split areas of the wall face of the blood vessel 100.

Next, as shown in FIG. 6, when switching the air supply switching valve 62 to the side of a common flow path of the split balloons B4, B5, and B6 and supplying air from the air supplying pump 60, air is delivered from the air supplying pump 60 to the split balloons B4, B5, and B6, thereby inflating the split balloons B4, B5, and B6 as shown in FIG. 7B. In addition, at this point, air is discharged via a discharge flow path from the heretofore inflated split balloons B1, B2, and B3, thereby causing the split balloons B1, B2, and B3 to be deflated. As shown in FIG. 7B, the deflated split balloons B1, B2, and B3 create gaps between themselves and the inner wall of the blood vessel 100, thereby securing blood flow.

The surfaces of the inflated split balloons B4, B5, and B6 are brought into close contact with the wall face of the blood vessel 100, and blood is removed from the close-contact portions to secure a field of view. At this point, in the same manner as described above, by rotating the optical fiber FB0 while emitting the measurement light beam L1 therefrom, as depicted in the right side of FIG. 8, images are required for every other area corresponding to portions with which the inflated (expanded) split balloons B4, B5, and B6 are in close contact among six circumferentially equally split areas of the wall face of the blood vessel 100.

A light beam reflected at each depth position of the wall face of the blood vessel 100 enters the optical fiber FB0 of the probe 30 as the reflecting light beam (returning light beam) L3, and is guided from the optical fiber FB0 to the optical fiber FB1 via the optical connector OC. Subsequently, the reflected light beam L3 is guided to the optical fiber FB6 via the optical bifurcator 34. The reflected light beam L3 guided by the optical fiber FB6 enters the optical multiplexing device 4.

Meanwhile, the reference light beam L2 split by the light splitting device 2 enters the optical path length adjusting device 20 from the optical fiber FB4 via the optical bifurcator 32 and the optical fiber FB5. The reference light beam L2 whose optical path length has been adjusted by the optical path length adjusting device 20 reenters the optical fiber FB5. Subsequently, the reference light beam L2 having entered the optical fiber FB5 is guided to the optical fiber FB7 via the optical bifurcator 32, and enters the optical multiplexing device 4 from the optical fiber FB7.

The optical multiplexing device 4 multiplexes the reflected light beam L3 reflected off of the wall face of the blood vessel 100 that is the measurement object S with the reference light beam L2 whose optical path length has been adjusted by the optical path length adjusting device 20.

Accordingly, the reflected light beam L3 and the reference light beam L2 are multiplexed and the interference light beams L4 and L5 are generated. The interference light beams L4 and L5 are detected as interference signals by the interference light detecting section 40 via the detectors 40a and 40b.

The detected interference signals are sent to the processing section 50. Upon acquisition of a transmitted interference signal, the processing section 50 acquires information regarding a measurement position from the optical connector OC and associates the interference signal with the positional information of the measurement position. Subsequently, at the processing section 50, as depicted at the center of FIG. 8, a depth-direction tomographic image of the entire circumference of the blood vessel 100 is created in which images acquired during inflation (expansion) of the split balloons B1, B2, and B3 are composited with images acquired during inflation (expansion) of the split balloons B4, B5, and B6. The generated tomographic image is transmitted to the displaying section 52 to be displayed.

As shown, in the present embodiment, by circumferentially splitting a transparent balloon into a plurality of balloons, inflating a part of the plurality of split balloons with air, and removing blood from portions at which a split balloon is in close contact with the inner wall of a blood vessel, it is now possible to capture a tomographic image of the inner wall of the blood vessel through the balloon. Meanwhile, at the split balloons not inflated, OCT imaging of the blood vessel can now be performed while securing blood flow and reducing the burden on the subject by not completely blocking blood flow.

Moreover, while a balloon has been split into six equal parts in the embodiment described above, the method of splitting a balloon is not limited thereto. For example, the balloon may be circumferentially split into four equal parts, whereby split balloons opposing each other across a center are to be inflated. Alternatively, the balloon may be split into an even greater number of split numbers. In this case, it is preferable to set the number of split balloons to an even number of four or more and perform imaging of a vascular wall by alternately inflating (expanding) and deflating every other split balloon because pressure will then be applied evenly in a circumferential direction of the inner wall of the blood vessel.

Furthermore, while a balloon has been inflated with air in the example described above, the material used for inflating the balloon need not be limited to air, and the balloon may instead be inflated by introducing a normal saline solution thereto, for example. In this case, the probe 30 is provided with a liquid supplying path in place of the air supplying path 66, and the optical tomographic imaging apparatus 1 is provided with a liquid supplying pump in place of the air supplying pump 60 and a liquid supply switching valve in place of the air supply switching valve 62. Then, after switching a split balloon to which a normal saline solution is to be supplied using the liquid supply switching valve, the normal saline solution is supplied to the split balloon to be inflated from the liquid supplying pump.

Although the optical tomographic imaging probe and the optical tomographic imaging apparatus using the same according to the present invention has been described in detail, it is obvious that the present invention is not limited to the examples shown above and that various changes and modifications can be made without departing from the scope thereof.

What is claimed is:

1. An optical tomographic imaging probe, comprising:
a tubular probe outer casing;
an optical fiber disposed inside the probe outer casing in an axial direction of the probe outer casing;
a plurality of transparent inflatable/deflatable split balloons provided circumferentially across an outer circumferential surface of a transparent portion of the probe outer casing, through which a light beam is to be emitted from the optical fiber towards an measurement object, so as to equally divide outer periphery area; and
a balloon inflating/deflating device that respectively and individually inflates/deflates each split balloon.

2. An optical tomographic imaging apparatus, comprising:
a light source that emits a light beam;
a light splitting device that splits the light beam emitted from the light source into a measurement light beam and a reference light beam;
an optical path length adjusting device that adjusts an optical path length of the reference light beam split by the light splitting device;
an irradiating optical system that irradiates the measurement light beam split by the light splitting device onto a measurement object;
an optical multiplexing device that multiplexes a reflected light beam from the measurement object when the measurement light beam is irradiated on the measurement object with the reference light beam;
an interference light detecting device that detects an interference light beam of the multiplexed reflected light beam and reference light beam; and
an image acquiring device that acquires a tomographic image of the measurement object from the detected interference light beam, wherein
the irradiating optical system includes the optical tomographic imaging probe according to claim 1, and
the optical tomographic imaging apparatus is arranged such that the optical tomographic imaging probe is inserted into a blood vessel as the measurement object, a portion of the plurality of split balloons are inflated to cause the outer surfaces of the inflated split balloons to come into close contact with an inner wall of the blood vessel to remove blood from the close-contact portions while split balloons other than the inflated split balloons are deflated so as to provide a gap between the outer surfaces of the deflated split balloons and the inner wall of the blood vessel to secure blood flow, the measurement light beam is irradiated onto the inner wall of the blood vessel via the split balloons in close contact with the inner wall of the blood vessel to acquire images of portions of the inner wall of the blood vessel in close contact with the outer surfaces of the inflated split balloons, switching is subsequently performed between the inflated split balloons and the deflated split balloons, images of portions of the inner wall of the blood vessel in close contact with the outer surfaces of split balloons inflated after the switching are acquired, and images obtained before and after the switching are composited by the image acquiring device to obtain an image of the entire circumference of the inner wall of the blood vessel.

3. The optical tomographic imaging apparatus according to claim 2, wherein the split balloons are split into an even number of split balloons of four or greater, the split balloons are divided into two groups respectively made up of every other split balloon in the circumferential direction, and the inflation/deflation of the split balloons is switched between the two groups by the balloon inflating/deflating device.

4. The optical tomographic imaging apparatus according to claim 2, wherein the balloon inflating/deflating device comprises: an air supplying pump that supplies air to each of the split balloons via an air supplying path provided in the optical tomographic imaging probe; and an air supply switching device that switches the supply of air to among the respective split balloons.

5. The optical tomographic imaging apparatus according to claim 3, wherein the balloon inflating/deflating device comprises: an air supplying pump that supplies air to each of the split balloons via an air supplying path provided in the optical tomographic imaging probe; and an air supply switching device that switches the supply of air to among the respective split balloons.

6. The optical tomographic imaging apparatus according to claim 2, wherein the balloon inflating/deflating device comprises: a liquid supplying pump that supplies a normal saline solution to each of the split balloons via a liquid supplying path provided in the optical tomographic imaging probe; and a liquid supply switching device that switches the supply of the normal saline solution to among the respective split balloons.

7. The optical tomographic imaging apparatus according to claim 3, wherein the balloon inflating/deflating device comprises: a liquid supplying pump that supplies a normal saline solution to each of the split balloons via a liquid supplying path provided in the optical tomographic imaging probe; and a liquid supply switching device that switches the supply of the normal saline solution to among the respective split balloons.
